(19) United States
(12) Patent Application Publication
(10) Pub. No.: US 2017/0251159 A1
(43) Pub. Date: Aug. 31, 2017

(54) METHOD AND SYSTEMS FOR DIAGNOSTIC MAPPING OF BLADDER

(71) Applicant: TARIS BIOMEDICAL LLC, Lexington, MA (US)

(72) Inventors: Hong Linh HO DUC, Weston, MA (US); Amit VASANJI, Pepper Pike, OH (US)

(21) Appl. No.: 15/511,820

(22) PCT Filed: Sep. 17, 2015
(86) PCT No.: PCT/US2015/050744

§ 371 Date: Mar. 16, 2017

Related U.S. Application Data
(60) Provisional application No. 62/051,879, filed on Sep. 17, 2014.

(51) Int. Cl. H04N 5/445; A61B 1/00; G06T 5/20; H04N 5/225; G06T 7/11; A61B 1/307; A61B 5/04

(52) U.S. Cl. CPC H04N 5/44504; A61B 1/307; A61B 1/00045; A61B 5/04; H04N 5/2256; G06T 7/11; G06T 5/20; H04N 2005/2255; G06T 2207/10016; G06T 2207/30024

(57) ABSTRACT

Methods and systems for generating a visualization of a surface of an internal body cavity, such as an internal organ like the bladder, are provided. The approach generally includes inserting an endoscope into an internal body cavity, acquiring a video of the tissue surfaces defining the internal body cavity, stitching video frames together to generate a panoramic map of the tissue surfaces defining the internal body cavity, and displaying the panoramic map.

### Cross-Reference to Related Applications

This application claims priority benefit of U.S. Provisional Application No. 62/051,879, filed Sep. 17, 2014, which is incorporated by reference herein.

### BACKGROUND

The present disclosure relates generally to the field of medical imaging, and more particularly methods and systems for diagnostic imaging of a surface of an internal body cavity.

Routinely used examinations of the bladder using a cystoscope can allow physicians to detect visible symptoms of conditions such as interstitial cystitis or bladder cancer. However, the data related to the features observed in the bladder are qualitative and subjective in nature, due to a lack of dimensional or color references in the bladder. Photographs or videos of the bladder surface can be acquired, but interpretation of the observations is left to the judgment of the physician, which can vary among individuals.

One result of this variability is that multiple readers are used in clinical trials to create a consensus opinion on visual data such as photographs or videos. This can make the process of tracking the progression of a disease and its visible symptoms difficult.

The introduction of quantitative measures such as dimensions (length, area) or color of bladder surface features would allow for more objective observations. In turn, these measures could ease the tracking of disease progression. However, absolute measurements are currently unattainable with conventional cystoscopy. This is because cystoscopes typically have an infinite focus distance, to allow for focused observation independent of distance from the bladder wall and to simplify the equipment at the head of the cystoscope. Without knowing the distance from the bladder wall at which an image is taken, one cannot deduce the dimensions of a feature without an internal reference in the picture. As for color, a white balance is typically performed prior to the cystoscopic procedure, but variation in the brightness of the light during examination due to auto-brightness settings can confound results. Manual adjustment of the light is impractical, as it would require constant readjustment by the operator, due to the changing needs for light intensity inside the bladder.

In a conventional approach, the bladder is mapped using a fixed length armature and rotation of an imaging sensor with known focal lengths and a priori defined motion to aid in panoramic stitching. However, this process is performed post-acquisition and requires re-insertion/re-imaging if a given region or frame is of low image quality. Furthermore, this process undesirably requires the use of specialized sensors and hardware (e.g., fluorescence or motorized cystoscopes) to carry out the imaging, and these may not be affordable or feasible for all clinical sites. In addition, re-imaging, for example due to failure points of such hardware, is not an option for patients with painful/sensitive bladder pathology.

Accordingly, there remains a need for improved methods and/or systems for imaging the bladder and making quantitative observations thereof It would be desirable to provide improved means for making quantitative observations about the bladder surface of a patient, for example in assessing the presence and/or progression (or regression) of a bladder disease.

### SUMMARY

In one aspect, a method for mapping an organ cavity is provided which includes inserting an endoscope into an organ cavity, wherein the organ cavity has tissue surfaces; acquiring a video of the tissue surfaces defining the organ cavity, wherein the video comprises a plurality of video frames; stitching the video frames together in real-time to generate a panoramic map of the tissue surfaces defining the organ cavity; and displaying the panoramic map.

In another aspect, a method is provided for mapping a urinary bladder of a patient from a video acquired of the urinary bladder via a cystoscope inserted into the bladder through the patient's urethra, wherein the method includes stitching together a plurality of video frames from the acquired video to generate a panoramic map of tissue surfaces defining the bladder; and displaying the panoramic map.

In still another aspect, a method is provided for tracking the progression of a disease or condition within a patient, wherein the method includes comparing a first panoramic map created at a first time with a second panoramic map created at a second time, the maps being created using any of the map generating methods disclosed herein.

In yet another aspect, a system is provided for mapping an organ cavity, which includes an endoscope; a video capture apparatus; an illumination device; a memory that stores computer-executable instructions, wherein the computer-executable instructions include instructions to: (i) receive a video of tissue surfaces defining the organ cavity, wherein the video comprises a plurality of video frames obtained with the video capture apparatus inserted into the organ cavity via the endoscope, the video being obtained while the tissue surfaces are illuminated by the illumination device; (ii) stitch the plurality of video frames together in real-time to generate a panoramic map of the tissue surfaces defining the organ cavity; and (iii) display the panoramic map; a processor configured to access the at least one memory and execute the computer-executable instructions; and a display screen, wherein the display screen is configured to display the panoramic map, e.g., in real-time.

### BRIEF DESCRIPTION OF DRAWINGS

Referring now to the drawings, which are meant to be exemplary and not limiting, and wherein like elements are numbered alike. The detailed description is set forth with reference to the accompanying drawings illustrating examples of the disclosure, in which use of the same reference numerals indicates similar or identical items. Certain embodiments of the present disclosure may include elements, components, and/or configurations other than those illustrated in the drawings, and some of the elements, components, and/or configurations illustrated in the drawings may not be present in certain embodiments.

FIG. 1 is a schematic of a system for diagnostic imaging of a surface of an internal body cavity.

FIG. 2 is a flowchart of a method for diagnostic imaging of a surface of an internal body cavity.

FIG. 3 is a flowchart of a method for processing a plurality of video frames.

FIG. 4 is a flowchart of a method for diagnosing or assessing disease progression.

### DETAILED DESCRIPTION

Systems and methods have been developed to provide unstructured, panoramic mapping of a tissue surface defining an internal body cavity. The systems and methods are based in part on the discovery that relative measurements can be obtained using substantially a whole internal body cavity surface as a reference for dimensions and color. The systems and methods are useful for, among other things, longitudinal evaluation of pathology. In a preferred embodiment, the panoramic mapping occurs in real-time or near real-time.

The devices and methods disclosed herein may be adapted for use in humans, whether male or female, adult or child, or for use in animals, such as for veterinary or livestock applications. Accordingly, the term “patient” may refer to a human or other mammalian subject.

While the methods and systems described in this application can be applied to any internal body cavity, a preferred embodiment will be described with reference to a urinary bladder. The urinary bladder is especially suited for the present systems and methods because conventional urinary bladder cystoscopy is performed in a manner that makes obtaining relative measurements difficult. Other representative examples of body cavities suitable for use with the methods and systems described herein include, without limitation, gastrointestinal tract cavities such as the esophagus, stomach, duodenum, small intestine, large intestine (colon), bile ducts, and rectum; respiratory tract cavities such as the nose or the lower respiratory tract; the ear; urinary tract cavities; and reproductive system cavities such as the cervix, uterus, and fallopian tubes.

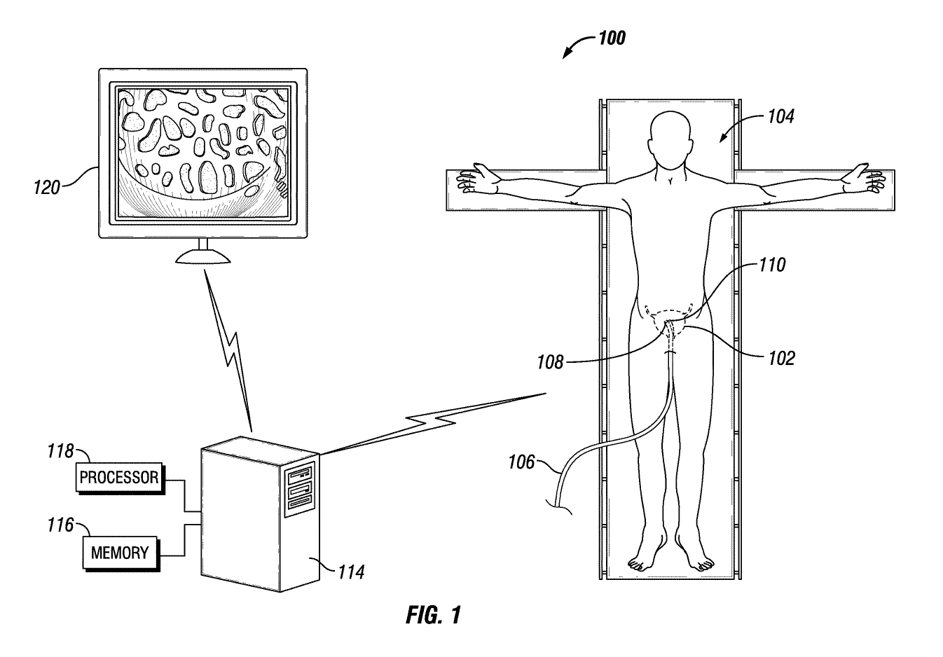

FIG. 1 is a schematic of a system 100 for diagnostic imaging of a surface of an internal body cavity 102 of a patient 104. As shown in FIG. 1, the system includes an endoscope 106. The endoscope 106 can be any device used to look inside an internal body cavity, such as a cystoscope.

The system 100 for diagnostic imaging also includes an image capture device 108. The image capture device 108 is used to acquire one or more images from an area of interest, e.g. the surface of an internal body cavity 102. The image capture device 108 can be any conventional device for capturing one or more images, such as a camera. The image capture device 108 may be partially or wholly integral to the endoscope 106, or partially or wholly coupled thereto. The image capture device 108 can also be free from the endoscope 106.

The system 100 for diagnostic imaging also includes an illumination device 110. The illumination device 110 is used to illuminate an area of interest, e.g. the surface of an internal body cavity 102. The illumination device 110 can be any conventional device. The illumination device 110 may be partially or wholly integral to the endoscope 106, or partially or wholly coupled thereto. The illumination device 110 can also be free from the endoscope 106. The illumination device 110 includes one or more light sources for illuminating an area of interest with an appropriate electromagnetic wavelength. Illustrative light sources include broadband or narrow band near infrared light sources, excitation laser sources, visible light sources, monochromatic light sources, other narrow band light sources, ultraviolet light sources, and the like. In one embodiment, the illumination device 110 includes a white light source covering the visible spectrum to faciliate the acquisition of a conventional color image.

The system 100 for diagnostic imaging is configured such that the image capture device 108 is in communication with a computer 114, which allows the output of the image capture device 108, e.g. one or more acquired images of the surface of an internal body cavity, to be received by the computer 114. The image capture device 108 can communicate with the computer 114 by any conventional means. For example, the image capture device 108 may communicate with the computer 114 by fiber optic cable, wirelessly, or through a wired or wireless computer network.

The computer 114 has a memory 116 and a processor 118. The memory 116 is capable of storing computer-executable instructions. The processor 118 is configured to access and execute computer-executable instructions stored in the memory 116. The computer-executable instructions may include, among other things, instructions for processing one or more received images, constructing a map from the received images, and displaying the map on a display device 120.

The system 100 for diagnostic imaging is configured such that the computer 114 is in communication with a display device 120, which allows an output of the computer 114, e.g. a map constructed from one or more images, to be received by the display device 120. The computer 114 can communicate with the display device 120 by any conventional means. For example, the computer 114 may communicate with the display device 120 by a video cable, wirelessly, or through a wired or wireless computer network.

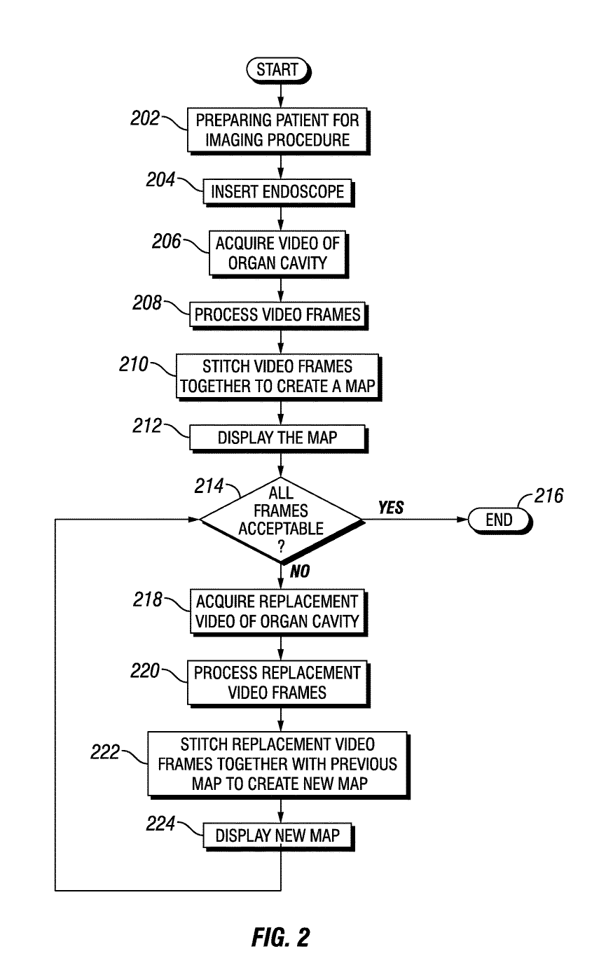

FIG. 2 is a flowchart of a method for diagnostic imaging of a surface of an internal body cavity. Step 202 prepares a patient for a diagnostic imaging procedure. This preparation can include conventional preparatory steps, such as sedating or anesthetizing a patient. In some embodiments, a patient is prepared for diagnostic imaging of a surface of a urinary bladder by draining the patient's urinary bladder entirely and then refilling the urinary bladder with a known volume of a sterile solution such as sterile saline or sterile water. The patient's urinary bladder may be drained and refilled by any other means known to those of skill in the art. For example, a urinary bladder may be drained by patient urination or through an inserted catheter, and a urinary bladder may be refilled using conventional bladder irrigation techniques. In some embodiments, the urinary bladder is refilled with a known volume of sterile solution after the bladder is drained, and the urinary bladder is kept at a known volume (e.g. a constant volume) during at least part of the diagnostic imaging procedure. Keeping the bladder at a constant volume advantageously allows for obtaining relative bladder measurements using substantially the whole bladder surface as a reference for dimensions and color.

In step 204, at least a portion of an endoscope, a portion of an image capture device, and a portion of an illumination device are inserted into an internal body cavity. The image capture device and the illumination device may be partially or wholly integral to the endoscope, or partially or wholly coupled thereto. The image capture device and the illumination device can also be free from the endoscope. In a preferred embodiment, the endoscope is a manually-guided conventional cystoscope with digital video image capture and white lighting.

In step 206, an image capture device acquires a video, or one or more images, that covers substantially an entire surface of an internal body cavity. As used herein, “substantially” an entire surface of an internal body cavity refers to about more than 80%, more than 85%, more than 90%, more than 95%, or more than 97.5% an entire surface of an internal body cavity.

In a preferred embodiment, a video of substantially an entire surface of an internal body cavity is acquired during a semi-unstructured cystoscopic evaluation of the bladder. A semi-unstructured cystoscopic evaluation is a cystoscopic evaluation wherein at least one, but not all, parameters of the evaluation are planned. For example, a semi-unstructured cystoscopic evaluation may have a predefined start point and predefined direction for panning the image capture device. In another example, a semi-unstructured cystoscopic evaluation may have a predefined start point and a number of other predefined points of interest. In this case, a physician locates and images the start point (e.g. a first point of interest) and then attempts to capture video of the other points of interest not using a predefined path for panning the image capture device.

In an alternative embodiment, a video of substantially an entire surface of an internal body cavity is acquired during a fully unstructured cystoscopic evaluation of the bladder. A fully unstructured cystoscopic evaluation is a cystoscopic evaluation wherein no parameters of the evaluation are planned. For example, a fully unstructured cystoscopic evaluation may have no predefined start point, no predefined points of interest, and no predefined path for panning the image capture device.

In an alternative embodiment, a video of substantially an entire surface of an internal body cavity is acquired during a fully structured cystoscopic evaluation of the bladder. A fully structured cystoscopic evaluation is a cystoscopic evaluation wherein all parameters of the evaluation are planned. For example, a fully structured cystoscopic evaluation may have a predefined start point, predefined points of interest, and a predefined path for panning the image capture device.

In some embodiments, a physician is provided with a display having information relevant to the cystoscopic evaluation procedure or, more particularly, the video acquisition step 206. For example, a display may show a blank map of an internal body cavity with predefined points of interest or features. The predefined points of interest or features can provide a frame of reference for use during cystoscopic evaluation of the internal body cavity and can be used as a reference for panning the image capture device in the internal body cavity to help ensure that a video of substantially an entire surface of the internal body cavity is acquired. For example, a display may show a representation of a bladder and a corresponding scan path. In some embodiments, points of interest correspond to regions of pathology or surface morphology (e.g. surface landmarks). The display may also include other relevant information. For example, the display may include example images or video to help guide a physician with respect to the imaging procedure. The display could also include useful information for the cystoscopic evaluation procedure or video acquisition step 206, such as information regarding the direction or path for panning the cystoscope, the image capture device, and the illumination device, the speed of cystoscope, image capture device, and illumination device movement, the brightness and contrast levels of light output by the illumination device, and the like.

In some embodiments, a physician locates and acquires an image at a first point of interest on the surface of the internal body cavity before acquiring images or video of the remaining surface of the internal body cavity. In some embodiments, a physician finds or locks onto a first point of interest on the surface of an internal body cavity and then pans the image capture device through the internal body cavity while attempting to pan over or capture video of other points of interest.

In step 208, the video or one or more images acquired in step 206 are received by a computer and processed. In a preferred embodiment, step 208 is performed contemporaneous with step 206. However, steps 206 and 208 may also be performed asynchronously. The processing step 208 may include any number of conventional methods used to process video or images that are known to those of skill in the art. In some aspects, the processing step 208 facilitates combining the video frames or images acquired in step 206 to form a map displayable on a display device. In some embodiments, processing step 208 includes feeding each acquired video frame or image into an algorithm that (1) unwarps each frame or image based on a known geometric transform, (2) extracts relevant feature information from each frame or image, (3) determines common feature points between each frame or image and other frames or images, and (4) computes homography between each frame or image and other frames or images.

In some embodiments, processing step 208 includes testing each acquired video frame or image for various image quality metrics. Each acquired video frame or image that fails to meet one or more quality metrics is deemed insufficient. Quality metrics are well-known to those of skill in the art. Exemplary quality metrics may include signal-to-noise ratio, image brightness, image contrast, low image quality, feature detection/matching failure, and the like.

Advantageously, a physician may be alerted that an acquired video frame or image is insufficient. For example, in one embodiment, an insufficient video frame or image is discarded and shown as an empty frame on a map of the surface of an internal body cavity (displayed on a display device). In this manner, a physician looking at the map will see empty regions on the map and know to rescan specific surfaces of the internal body cavity corresponding to blank regions on the map in order to acquire replacement video or images. Alternatively, a physician can discard all captured video or images and completely restart the image acquisition procedure.

In step 210, processed video frames or images are stitched together to create a map of the surface of an internal body cavity. In some embodiments, the video frames or images are stitched together to form a two dimensional map projection. In this way, dimensions can be expressed in relation to the total internal body cavity surface. The map projection can be any suitable projection, such as a cylindrical projection (i.e., a Mercator projection). In some embodiments, the video frames or images are stitched together to create a panoramic map of the bladder. Stitching can be scale and rotationally agnostic. Further, stitched maps can incorporate predefined internal body cavity surface morphology. In a preferred embodiment, step 210 is performed contemporaneous with steps 206 and 208. However, step 210 may also be performed asynchronously from steps 206 and 208.

In certain embodiments, each video frame or image is only stitched with the preceding video frame or image. The first video frame or image may or may not be stitched with a blank map of an internal body cavity surface having points of interest or features (e.g. surface morphology) displayed thereon. However, in some embodiments, if a video frame or image overlaps not only the preceding video frame or image but also other existing video frame(s) or image(s), then the video frame or image is stitched with all video frames or images with which it overlaps. This ensures accurate placement of each video frame or image in relation to all other overlapping video frames or images. In certain embodiments, each video frame or image deemed insufficient by processing step 208 is displayed as a bank region on the map.

In step 212, a stitched map from step 210 is displayed on a display device. In a preferred embodiment, step 212 is performed contemporaneous with steps 206, 208, and 210. However, step 212 may also be performed asynchronously from steps 206, 208, and 210. Performing step 212 contemporaneously with video acquisition step 206, processing step 208, and stitching step 210 advantageously allows for a physician to not only discern what internal body cavity surface areas have been imaged by looking at the map, but also affords the physician the ability to rescan internal body cavity surface areas that were previously scanned but yielded insufficient video frames or images, which may be shown as blank regions on the map. In a preferred embodiment where a stitched result and a display showing the same are continuously and immediately updated with newly acquired video frames or images, a physician can rescan an empty region by simply retracing his or her path of video or image acquisition.

In step 214, a physician determines whether the displayed map of an internal body cavity surface is acceptable. If, for example, the map is substantially complete and composed of good quality images or video frames, a physician may accept the map (i.e. the map is finalized) and the method for diagnostic imaging of a surface of an internal body cavity is ended 216. Alternatively, if the map is not substantially complete and/or not composed of good quality images or video frames, but still suffices for diagnostic use, a physician may accept the map (i.e. the map is finalized) and the method for diagnostic imaging of a surface of an internal body cavity is ended 216. However, if the map is not substantially complete, not composed of good quality images or video frames, or includes blank regions corresponding to insufficient video frames or images, a physician may not accept the map. In this case, the physician moves on to the next step.

In step 218, an image capture device acquires replacement video, or one or more images, to replace any video frames or images that need replacing. Step 218 is carried out in substantially the same manner as step 206 except that step 218 may only require panning the image capture device through less than substantially an entire surface of an internal body cavity, owing to the fact that only certain video frames or images may need replacing.

In step 220, the replacement video or one or more images acquired in step 218 are received by a computer and processed. Step 220 is carried out in substantially the same manner as step 208.

In step 222, processed replacement video frames or images from step 220 are stitched together with each other and previously stitched frames to create an updated map of the surface of an internal body cavity. Step 222 is carried out in substantially the same manner as step 210.

In step 224, the updated map from step 222 is displayed on a display device. Step 224 is carried out in substantially the same manner as step 212. Once displayed, a physician moves back to step 214 to determine whether the updated map of an internal body cavity surface is acceptable.

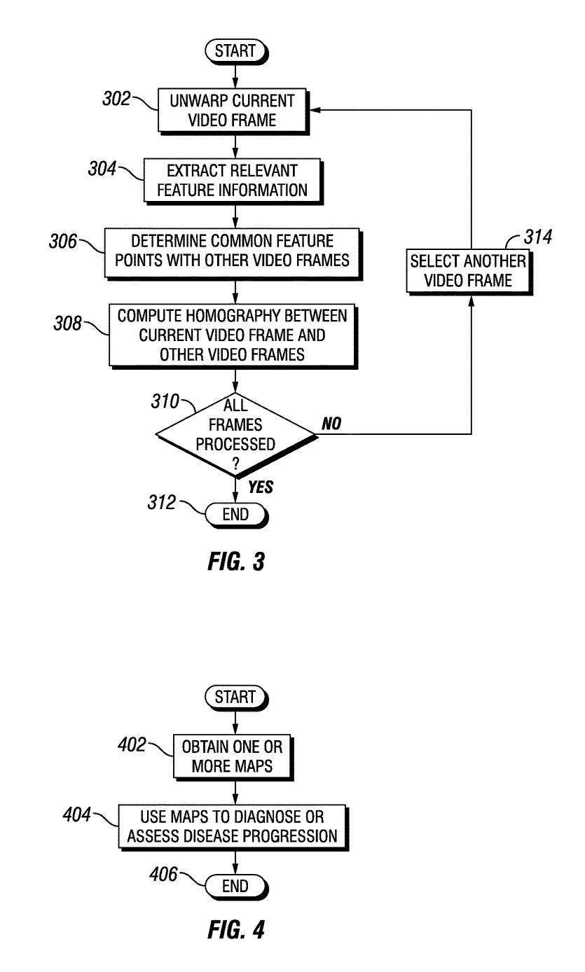

FIG. 3 is a flowchart of a method for processing a plurality of video frames or images. In step 302, a video frame or image is fed into an algorithm that unwarps the frame or image. A video frame or image can be unwarped using any means known to those of skill in the art.

In step 304, a video frame or image is fed into an algorithm that extracts relevant feature information (e.g., blood vessels) from the video frame or image. Relevant feature information can be extracted using any means known to those of skill in the art. In some embodiments, a spectral based filter is used to extract relevant feature information from a video frame or image.

In step 306, a video frame or image is fed into an algorithm that determines common feature points between the current video frame or image and other processed video frames or images. Determining common feature points between the current video frame or image and other processed video frames or images can be done using any means known to those of ordinary skill in the art. In some embodiments, a scale-invariant feature transform (SIFT) or a Harris corner detector is used to determines common feature points between the current video frame or image and other processed video frames or images.

In step 308, a video frame or image is fed into an algorithm that computes homography between the current video frame or image and other processed video frames or images, eliminates outliers, and generates a transform for stitching the current video frame or image with other processed video frames or images. Computing homography, eliminating outliers, and generating a transform for image stitching can be done using any means known to those of skill in the art. In some embodiments, homography between the current video frame or image and other processed video frames or images is computed using a Random Sample Consensus (RANSAC) algorithm to narrow the number of SIFT descriptors.

In step 310, an algorithm determines whether all captured video frames or images have been processed. If the answer is yes, the method for processing a plurality of video frames or images is ended 312. If the answer is no, a new video frame or image is selected 314 and supplied to step 302.

FIG. 4 is a flowchart of a method for diagnosing or assessing disease progression. In step 402, one or more maps of substantially an entire surface of an internal body cavity are obtained using any of the methods or systems disclosed herein. In some embodiments, one or more maps are registered to a patient. In this manner, the maps of substantially an entire surface of an internal body cavity are associated with a particular patient. When there are two or more maps associated with a particular patient, the maps can be comparatively analyzed.

In some embodiments, mapping of an internal body cavity is carried out periodically, such as weekly, bi-weekly, monthly, annually, and the like. In some embodiments where a disease is treated, for example with a therapeutic agent (e.g., a drug), mapping of an internal body cavity can be carried out prior to treatment with the therapeutic agent, during the therapeutic treatment, and after concluding the therapeutic treatment.

In step 404, the one or more maps are used to diagnose, assess, or track the progression of a disease. In some embodiments, a single map is used to diagnose or assess the progression of a disease. In a preferred embodiment, two or more maps are compared against one another to diagnose, assess, or track the progression of a disease. The two or more maps can be compared against one another by any suitable means. For example, a physician may locate a specific region or regions of interest (e.g. regions of pathology) on each map and evaluate any observed differences between the region or regions of interest on the two or more maps. The map comparison process can be utilized for, among other things, longitudinal/temporal evaluation of pathology at specific regions of the surface of an internal body cavity, such as the bladder, to assess a response to therapeutic intervention or monitor disease progression.

The map comparison may include comparing the size and/or number of areas within the map that include an observable characteristic of the urothelium, for example. For instance, the observable characteristic could be a lesion, inflammation, or the like.

Computers can assist with diagnosing, assessing, and tracking the progression of a disease or comparing maps against one another using any number of means readily recognizable to those of skill in the art. For example, computer algorithms can align or overlay two or more maps using points of interest, such as regions of pathology or surface morphology (e.g. surface landmarks). Using a computer algorithm to align or overlay two or more maps, once validated with physician input, advantageously provides consistency to the diagnosing, assessing, and tracking process by removing some subjectivity associated with human manipulation. Likewise, computer algorithms can detect changes in points of interest, e.g. size, coloration, and the like, which also facilitates diagnosing, assessing, or tracking the progression of a disease by removing some subjectivity associated with human reading of the maps.

In some embodiments, two or more maps are compared against one another to evaluate the effectiveness of a selected therapeutic treatment on a patient in need of treatment for a disease, such as Hunner's lesions or bladder cancer. For example, mapping can be carried out periodically pre-treatment, during treatment, and post-treatment, and then the maps compared to quantitatively assess whether visible lesions or tumors are responding to a selected therapeutic treatment (e.g. whether lesions or tumors are reduced in size). This information can be useful for a number of purposes, including measuring therapeutic effectiveness or tolerability of a drug in a clinical trial. Further, when tracking the progression of a disease such as cancer, quantitative data can be normalized to the total surface of an internal body cavity (e.g. a bladder) each time a patient is assessed in order to provide comparable data. In some embodiments, the background color of a surface of an internal body cavity (e.g. bladder) is used as a baseline and changes in coloration are analyzed. In some embodiments, the size and shape of surface morphology in regions of interest are followed, and change in size and shape are analyzed. After the maps are used to diagnose or assess disease progression/status, the method is ended 406.

The techniques described herein can be used to map a variety of different internal body cavity surfaces including, but not limited to, the bladder. For example, the techniques may be applied to any endoscopic procedure where a scan trajectory could be defined, where tissue is not actively manipulated while scanning and stitching, and where features within the scan field can be sufficiently and clearly distinguished.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

1. A method for mapping an organ cavity, comprising:
inserting an endoscope into an organ cavity, wherein the organ cavity has tissue surfaces;
acquiring a video of the tissue surfaces defining the organ cavity, wherein the video comprises a plurality of video frames;
stitching the video frames together in real-time to generate a panoramic map of the tissue surfaces defining the organ cavity; and
displaying the panoramic map.

2. The method of claim 1, wherein the organ cavity is a urinary bladder and the video captures substantially all of the tissue surfaces defining the urinary bladder.

3. The method of claim 1, wherein the endoscope is a manually guided cystoscope with video image capture and lighting.

4. The method of claim 1, further comprising draining a first fluid from the organ cavity and then filling the organ cavity with a known volume of a second fluid, and wherein the organ cavity maintains a nearly constant volume during the acquiring step.

5. The method of claim 1, wherein the panoramic map is a two-dimensional cylindrical projection.

6. The method of claim 1, wherein the acquiring step is done in a semi-unstructured manner and comprises:
locating a first point of interest on the tissue surfaces of the organ cavity;
obtaining a first video frame at the first point of interest;
panning through the organ cavity; and
obtaining one or more additional video frames at one or more other points of interest on the tissue surfaces of the organ cavity.

7. The method of claim 6, further comprising processing each of the plurality of video frames, wherein the processing comprises:
unwarping each of the plurality of video frames;
applying a spectral based filter to extract relevant feature information from each of the plurality of video frames;
applying a scale-invariant feature transform or a Harris corner detector to each of the plurality of video frames to determine common feature points between each of the plurality of video frames and at least one other video frame; and
computing homography between each of the plurality of video frames and at least one other video frame using a random sample consensus algorithm to narrow a number of the scale-invariant feature transform descriptors, eliminate outlier scale-invariant feature transform descriptors, and generate a transform for image stitching.

8. The method of claim 7, wherein at least some of the plurality of video frames are stitched and displayed on the panoramic map while the panning step is ongoing.

9. The method of claim 8, wherein the panoramic map includes blank regions corresponding to each of the plurality of video frames that either have low image quality or failed to generate a transform for image stitching.

10. The method of claim 9, wherein the acquiring, processing, stitching, and displaying steps are optionally repeated for one or more sections of the organ cavity corresponding to each of the plurality of video frames that either have low image quality or failed to generate a transform.

11. The method of claim 1, wherein the panoramic map is initially blank and includes one or more predefined points of interest.

12. The method of claim 1, further comprising processing each of the plurality of video frames, wherein the acquiring step is fully unstructured and the processing step comprises:
unwarping each of the plurality of video frames;
applying a spectral based filter to extract relevant feature information from each of the plurality of video frames;
applying a scale-invariant feature transform or a Harris corner detector to each of the plurality of video frames to determine common feature points between each of the plurality of video frames and at least one other video frame; and
computing homography between each of the plurality of video frames and at least one other video frame using a random sample consensus algorithm to narrow a number of the scale-invariant feature transform descriptors, eliminate outlier scale-invariant feature transform descriptors, and generate a transform for image stitching.

13. The method of claim 3, further comprising processing each of the plurality of video frames, and wherein the substantially captured entire surface of the bladder is used as a reference for processing, stitching, or displaying the map.

14. The method of claim 13, wherein the relevant feature information comprises a color, and wherein the substantially captured entire surface of the bladder has a baseline background color.

15. A method for mapping a urinary bladder of a patient from a video acquired of the urinary bladder via a cystoscope inserted into the bladder through the patient's urethra, comprising:
stitching together a plurality of video frames from the acquired video to generate a panoramic map of tissue surfaces defining the bladder; and
displaying the panoramic map.

16. The method of claim 15, wherein the stitching and displaying steps are conducted in real-time with acquisition of the video.

17. A method for tracking the progression of a disease or condition within a patient, comprising:
comparing a first panoramic map created at a first time according to the method of claim 1 with a second panoramic map created at a second time according to the method of claim 1.

18. The method of claim 17, wherein the organ cavity is the urinary bladder of the patient.

19. The method of claim 18, further comprising using a result of said comparing to assess the effectiveness or tolerability of a therapeutic treatment administered to the patient for the disease or condition.

20. The method of claim 19, wherein the disease or condition comprises Hunner's lesions or bladder cancer.

21. A system for mapping an organ cavity, comprising:
an endoscope;
a video capture apparatus;
an illumination device;
a memory that stores computer-executable instructions, wherein the computer-executable instructions comprise instructions to:
receive a video of tissue surfaces defining the organ cavity, wherein the video comprises a plurality of video frames obtained with the video capture apparatus inserted into the organ cavity via the endoscope, the video being obtained while the tissue surfaces are illuminated by the illumination device;
stitch the plurality of video frames together in real-time to generate a panoramic map of the tissue surfaces defining the organ cavity; and
display the panoramic map;
a processor configured to access the at least one memory and execute the computer-executable instructions; and
a display screen, wherein the display screen is configured to display the panoramic map in real-time.

22. The system of claim 21, wherein the computer-executable instructions further comprise instructions to process each of the plurality of video frames, wherein the instructions to process each of the plurality of video frames comprise:
unwarp each of the plurality of video frames;
apply a spectral based filter to extract relevant feature information from each of the plurality of video frames;
apply a scale-invariant feature transform or a Harris corner detector to each of the plurality of video frames to determine common feature points between each of the plurality of video frames and at least one other video frame; and
compute homography between each of the plurality of video frames and at least one other video frame using a random sample consensus algorithm to narrow a number of the scale-invariant feature transform descriptors, eliminate outlier scale-invariant feature transform descriptors, and generate a transform for image stitching.

23. The system of claim 21, wherein the computer-executable instructions further comprise instructions to stitch at least some of the plurality of video frames together and display the panoramic map while at least some of the video frames are still being captured.

24. The system of claim 21, wherein the panoramic map is a two-dimensional cylindrical projection.

25. The system of claim 21, wherein the map is initially blank and includes one or more predefined points of interest.

26. The system of claim 21, wherein the panoramic map is configured to allow for blank regions corresponding to each of the plurality of video frames that either have low image quality or failed to generate a transform for image stitching.

27. The system of claim 21, wherein the computer-executable instructions further comprise instructions to optionally receive, process, stitch, and display additional video frames for one or more sections of the organ cavity corresponding to each of the plurality of video frames that either have low image quality of failed to generate a transform.

28. The system of claim 21, wherein the endoscope comprises a cystoscope configured for passage through the urethra of a patient.
